(19) United States
(12) Patent Application Publication
(10) Pub. No.: US 2016/0051148 A1
(43) Pub. Date: Feb. 25, 2016

(54) Optoacoustic Imaging Device

(71) Applicant: XTrillion, Inc., Tokyo (JP)

(72) Inventor: Naoto SATO, Tokyo (JP)

(21) Appl. No.: 14/753,372

(22) Filed: Jun. 29, 2015

(30) Foreign Application Priority Data
Aug. 20, 2014 (JP) 2014-167685

(51) Int. Cl. A61B 5/00; A61B 5/0402

(52) U.S. Cl. CPC A61B 5/0095; A61B 5/0402; A61B 5/7292

(57) ABSTRACT

An optoacoustic imaging device has a light source module which irradiates a tested object with light, a light source driver which drives and controls the light source module, a detector which detects an optoacoustic wave generated inside the tested object as a result of the tested object being irradiated with the light, an image generator which generates still image information based on a detection signal from the detector, and an acquirer which acquires an organ pulsation signal. The organ pulsation signal is used as a trigger to make the light source driver drive the light source module and to make the image generator generate the still image information.

This application is based on Japanese Patent Application No. 2014-167685 filed on Aug. 20, 2014, the contents of which are hereby incorporated by reference.

### BACKGROUND OF THE INVENTION

1. Field of the Invention

The present invention relates to optoacoustic imaging devices.

2. Description of Related Art

Conventionally, as devices for acquiring cross-sectional images inside a living body, there are known ultrasonic imaging diagnosis devices. Ultrasonic imaging diagnosis devices are capable of transmitting an ultrasonic wave into a living body as a tested object, performing luminance modulation on the reflection signal of the ultrasonic wave, and displaying cross-sectional morphological images. Some devices are capable of exploiting the Doppler effect to display blood velocity distribution, and some modern devices are even capable of displaying tissue elasticity.

On the other hand, in recent years, there has been developed optoacoustic imaging technology. In optoacoustic imaging technology, a living body as a tested object is irradiated nub pulsating light from a laser or the like. Then a living tissue inside the living body absorbs the pulsating light, and as a result of adiabatic expansion, an optoacoustic wave (ultrasonic wave), which is an elastic wave, is generated. This optoacoustic wave is detected with an ultrasonic probe, an optoacoustic image is generated based on the detection signal, and thereby the interior of the living body is visualized. By using pulsating light of a wavelength in or around a near-infrared region, it is possible to visualize differences in composition between different living tissues, for example differences in the amount of hemoglobin, the degree of oxidation, the amount of lipids, etc.

In analysis and diagnosis of a pathologically affected part, blood flow distribution and the pulsatility of blood flowing into the affected pan are observed to determine, for example, malignity. If pulsatility is present, blood flow increases in cardiac systole and decreases in cardiac diastole. One approach is to acquire moving image information on the affected part, but this requires storage and playback of moving images, leading to an increased amount of data stored and an increased analysis time.

With the optoacoustic imaging mentioned above, it is possible to grasp blood flow itself in the affected part, but as to its relationship with heart beats, it is necessary to separately test the heart, and thus a user has to conduct analysis on the acquired rest results, leading to an increased analysis time.

Incidentally, Japanese patent application published No. 2001-292993 discloses an ultrasonic diagnosis device that generates an ultrasonic cross-sectional image in synchronism with an electrocardiographic signal, but suggests nothing about optoacoustic imaging.

### SUMMARY OF THE INVENTION

An object of the present invention is to provide an optoacoustic imaging device that allows a user easy analysis of information acquired from an optoacoustic wave for study in relation to organ pulsation (e.g., heart beats).

To achieve the above object, according to the present invention, an optoacoustic imaging device includes: a light source module which irradiates a tested object with light; a light source driver which drives and controls the light source module; a detector which detects an optoacoustic wave generated inside the tested object as a result of the tested object being irradiated with the light; an image generator which generates still image information based on a detection signal from the detector, and an acquirer which acquires an organ pulsation signal. Here, the organ pulsation signal is used as a trigger to make the light source driver drive the light source module and to make the image generator generate the still image information (a first configuration).

In the first configuration described above, the image generator may generate the still image information only at first and second timings within one cycle of the organ pulsation signal, the first tinting corresponding to systole of an organ and the second timing corresponding to diastole of an organ (a second configuration).

With this configuration, it is possible to acquire images appropriate for study in relation to organ pulsation while greatly reducing the amount of data.

In the second configuration described above, the first timing may be a timing delayed by a first delay time from the timing at which a predetermined wave indicating contraction of the organ is detected in the organ pulsation signal, and the second timing may be a timing delayed by a second delay time, which is longer than the first delay time, from the timing at which the predetermined wave is detected in the organ pulsation signal (a third configuration).

With this configuration, it is possible to acquire images with consideration given to a delay in issue reaction inside the tested object.

In the first configuration described above, during a predetermined period from a timing delayed by a predetermined delay time from the timing at which the predetermined wave is detected in the organ pulsation signal, the image generator may generate a plurality of sets of still image information.

With this configuration, it is possible to acquire appropriate images even when the delay in tissue reaction varies from one tested object to another.

### BRIEF DESCRIPTION OF THE DRAWINGS

FIG. 1A is a schematic exterior view of an optoacoustic imaging device embodying the present invention;

FIG. 1B is a block configuration diagram of an optoacoustic imaging device embodying the present invention;

FIG. 2A is a schematic front view of an ultrasonic probe embodying the present invention;

FIG. 2B is a schematic side view of an ultrasonic probe embodying the present invention;

FIG. 3 is a diagram showing an example of arrangement of LED elements in a light source module included in an ultrasonic probe embodying the present invention;

FIG. 4 is a timing chart in connection with synchronous electrocardiographic imaging according to a first embodiment of the present invention; and

FIG. 5 is a timing chart in connection with synchronous electrocardiographic imaging according to a second embodiment of the present invention.

### DETAILED DESCRIPTION OF PREFERRED EMBODIMENTS

### First Embodiment

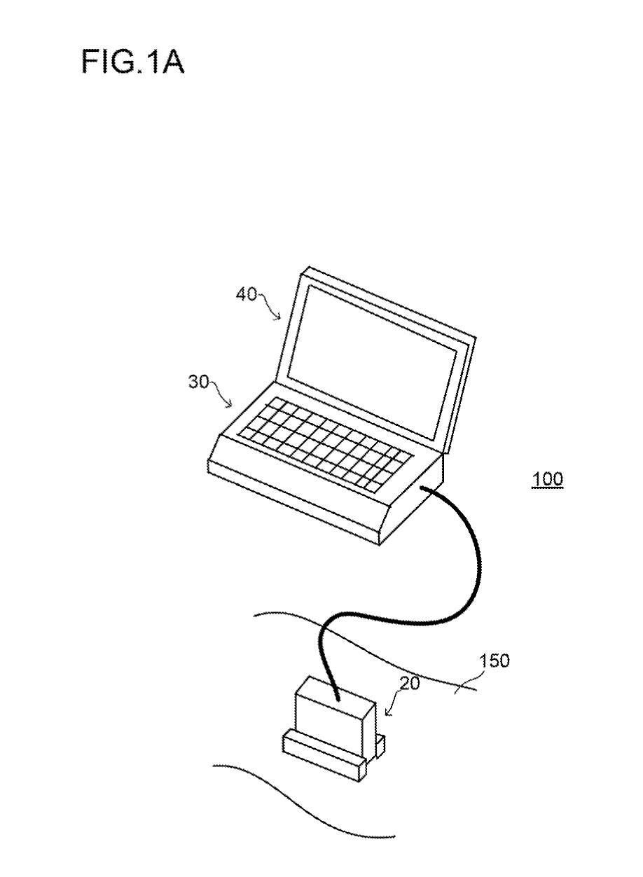

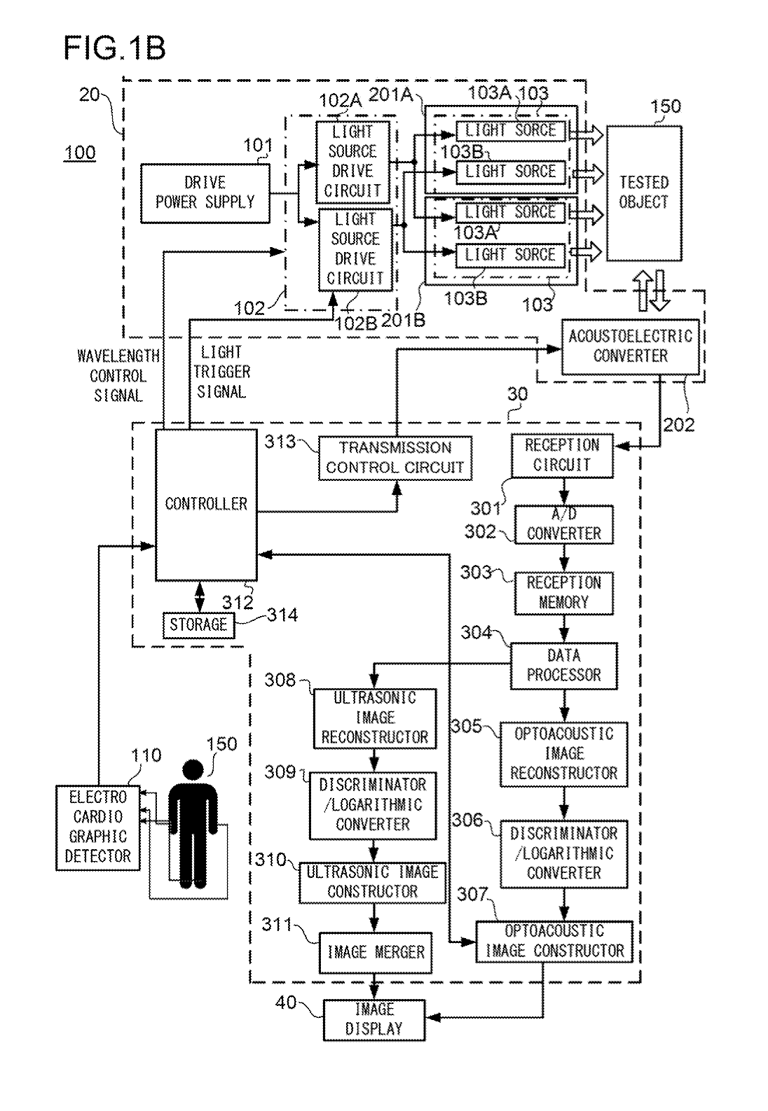

Hereinafter, embodiments of the present invention will be described with reference to the accompanying drawings. First, with reference to FIGS. 1A to 3, the configuration Fig. an optoacoustic imaging device according to a first embodiment of the present invention will be described.

FIG. 1A is a schematic exterior view of the optoacoustic imaging device 100. The optoacoustic imaging device 100 includes an ultrasonic probe 20 for acquiring cross-sectional image information from inside a tested object 150, an image generator 30 for processing the signal detected by the ultrasonic probe 20 to turn it into an image, and an image display 40 for displaying the image generated by the image generator 30.

More specifically, as shown in FIG. 1B, the optoacoustic imaging device 100 includes an ultrasonic probe 20 which irradiates the tested object 150, which is a living body, with light and detects an optoacoustic wave generated inside the tested object 150, and an image generator 30 which generates an optoacoustic image based on a detection signal of the optoacoustic wave. The ultrasonic probe 20 also transmits an ultrasonic wave into the tested object 150 and detects the reflected ultrasonic wave. The image generator 30 also generates an ultrasonic image based on a detection signal of the ultrasonic wave. The optoacoustic imaging device 100 further includes an image display 40 which displays an image based on an image signal generated by the image generator 30.

The ultrasonic probe 20 includes a drive power supply 101, a light source driver 102 which is supplied with electric power from the drive power supply 101, an irradiator 201A, an irradiator 201B, and an acoustoelectric converter 202. The irradiators 201A and 201B each include a light source module 103. Each light source module 103 includes light sources 103A and 103B, which are LED light sources. The light source driver 102 includes a light source drive circuit 102A, which drives the light source 103A, and a light source drive circuit 102B, which drives the light source 103B.

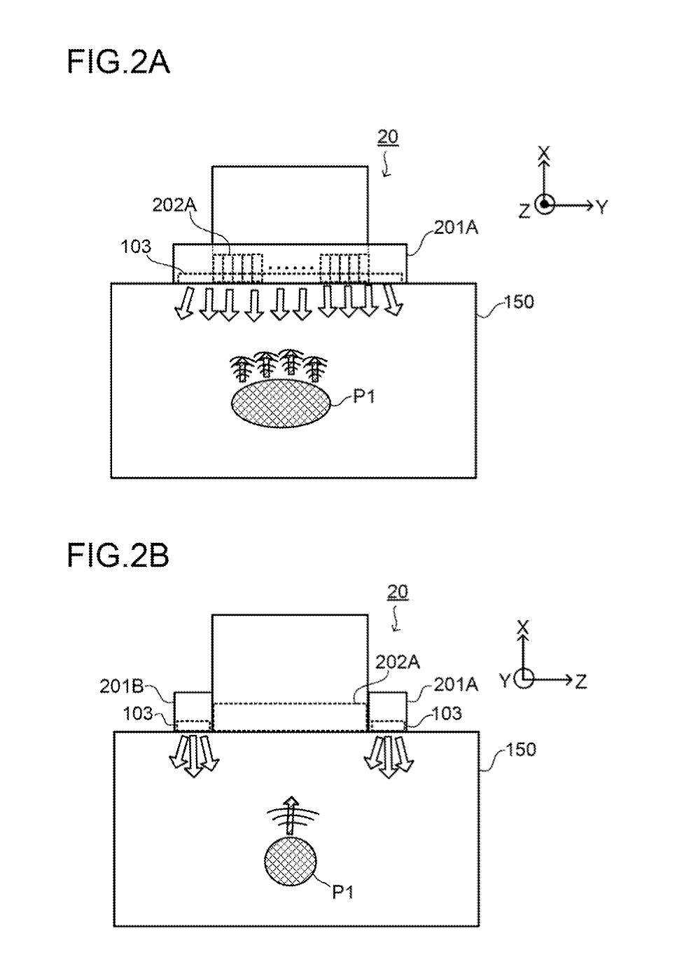

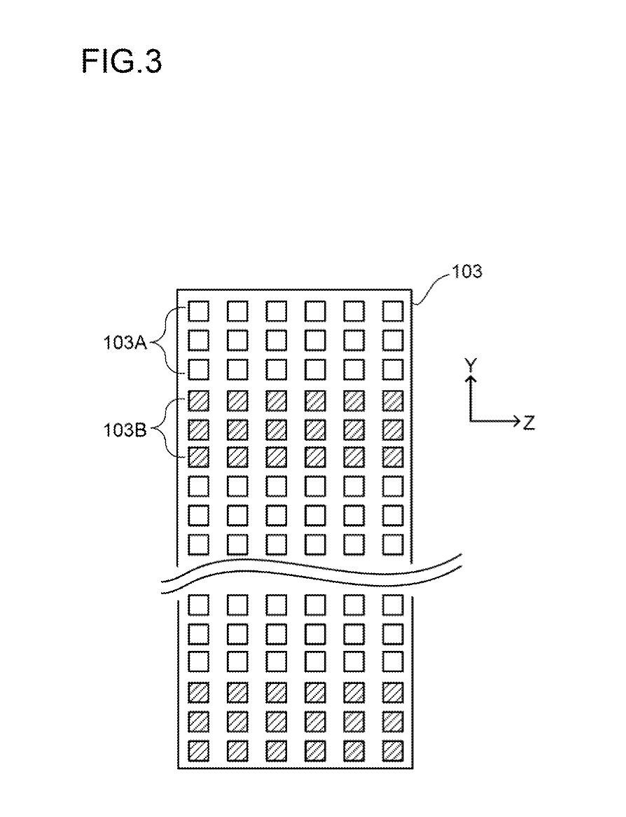

A schematic from view and a schematic side view of the ultrasonic probe 20 are shown in FIGS. 2A and 2B respectively. As shown in FIGS. 2A and 2B, the irradiators 201A and 201B are arranged opposite each other in the Z direction. An example of the arrangement of light sources in the light source module 103 provided in each of the irradiators 201A and 201B is shown in FIG. 3. In the example shown in FIG. 3, the light source module 103 has light sources 103A and light sources 103B arranged alternately in the Y direction, the light sources 103A and 103B each being composed of LED elements in three rows in the Y direction and six rows in the Z direction. In each of the irradiators 201A and 201B, the light source module 103 is so arranged as to be located close to the tested object 150 when the ultrasonic probe 20 is put in contact with the tested object 150.

Between the light sources 103A and 103B, the LED elements have different emission wavelengths. The light source drive circuit 102A (FIG. 1B) makes the LED elements of the light sources 103A in the irradiators 201A and 201B emit light, so that the tested object 150 is irradiated with the light. Likewise, the light source drive circuit 102B makes the LED elements of the light sources 103B in the irradiators 201A and 201B emit light, so that the tested object 150 is irradiated with the light.

The irradiators 201A and 201B shown in FIGS. 2A and 2B may be configured to include, for example, a lens for converging the light from the LED light sources shown in FIG. 3, and further a light guide made of acrylic resin or the like for guiding the light converged by the lens to the tested object. The light sources are not limited to LED light sources; for example, in a case where laser light sources (comprising semiconductor laser elements) are used, an optical fiber may be provided through which to guide laser light emitted from the laser light sources provided externally to the probe to the irradiators 201A and 201B. For another example, the light source module may be composed of organic light-emitting diode elements.

The acoustoelectric converter 202 is composed of a plurality of ultrasonic oscillating elements 202A arranged in the Y direction between the irradiators 201A and 201B. The ultrasonic oscillating elements 202A are piezoelectric elements which, when a voltage is applied to them, oscillate and generate an ultrasonic wave and which, when vibration (ultrasonic wave) is applied to them, generate voltage. Between the acoustoelectric converter 202 and the surface of the tested object 150, an adjustment layer (unillustrated) is provided which allows adjustment of a difference in acoustic impedance. The adjustment layer serves to propagate the ultrasonic wave generated by the ultrasonic oscillating elements 202A efficiently into the tested object 150, and also serves to propagate the ultrasonic wave (including an optoacoustic wave) from inside the tested object 150 efficiently to the ultrasonic oscillating elements 202A.

The irradiators 201A and 201B emit pulsating light, which enters the tested object 150 while being diffused, and is absorbed by a light absorber (living tissue) inside the tested object 150. When the light absorber (e.g., living tissue P1 shown in FIGS. 2A and 2B) absorbs light, adiabatic expansion occurs, whereby an optoacoustic wave (ultrasonic wave), which is an elastic wave, is generated. The generated optoacoustic wave propagates inside the tested object 150, and is converted into a voltage signal by the ultrasonic oscillating elements 202A.

The ultrasonic oscillating elements 202A also generate an ultrasonic wave to transmit it into the tested object 150, and receives the ultrasonic wave reflected inside the tested object 150 to generate a voltage signal. Thus, the optoacoustic imaging device 100 of this embodiment can perform not only optoacoustic imaging but also ultrasonic imaging.

The image generator 30 (FIG. 1B) includes a reception circuit 301, an A/D converter 302, a reception memory 303, a data processor 304, an optoacoustic image reconstructor 305, a discriminator/logarithmic converter 306, an optoacoustic image constructor 307, an ultrasonic image reconstructor 308, a discriminator/logarithmic converter 309, an ultrasonic image constructor 310, an image merger 311, as controller 312, a transmission control circuit 313, and a storage 314.

The reception circuit 301 selects, out of the plurality of ultrasonic oscillating elements 202A, a part of them, and amplifies the voltage signal (detection signal) with respect to the selected ultrasonic oscillating elements.

In optoacoustic imaging, for example the plurality of ultrasonic oscillating elements 202A are divided into two regions adjoining in the Y direction; of the two regions, one is selected for first-time irradiation, and the other is selected for second-time irradiation. In ultrasonic imaging, for example, an ultrasonic wave is generated while switching is performed from one part of the plurality of ultrasonic oscillating elements 202A to another, i.e., from one group of adjoining ultrasonic oscillating elements to another (so-called linear electronic scanning), and the reception circuit 301 accordingly so switches as to select one group after another.

The A/D convener 302 converts the amplified detection signal from the reception circuit 301 into a digital signal. The reception memory 303 stores the digital signal from the A/D converter 302. The data processor 304 serves to branch the signal stored in the reception memory 303 between the optoacoustic image reconstructor 305 and the ultrasonic image reconstructor 308.

The optoacoustic image reconstructor 305 performs phase matching addition based on the detection signal of an optoacoustic wave, and reconstructs the data of the optoacoustic wave. The discriminator/logarithmic converter 306 performs logarithmic compression and envelope discrimination on the data of the reconstructed optoacoustic wave. The optoacoustic image constructor 307 then converts the data that has undergone the processing by the discriminator/logarithmic converter 306 into pixel-by-pixel luminance value data. Specifically, according to the amplitude of the optoacoustic wave, optoacoustic image data (grayscale data) is generated as data comprising the luminance value at every pixel on the XY plane in FIG. 2A.

On the other hand, the ultrasonic image reconstructor 308 performs phase matching addition based on the detection signal of an ultrasonic wave, and reconstructs the data of the ultrasonic wave. The discriminator/logarithmic converter 309 performs logarithmic compression and envelope discrimination based on the data of the reconstructed ultrasonic wave. The ultrasonic image constructor 310 then converts the data that has undergone the processing by the discriminator/logarithmic converter 309 into pixel-by-pixel luminance value data. Specifically, according to the amplitude of the ultrasonic wave as the reflected wave, ultrasonic image data (grayscale data) is generated as data comprising the luminance value at every pixel on the XY plane in FIG. 2A. Display of a cross-sectional image through transmission and reception of an ultrasonic wave as described above is generally called B-mode display.

The image merger 311 merges the optoacoustic image data and the ultrasonic image data together to generate composite image data. The image merging here may be achieved by superimposing the optoacoustic image on the ultrasonic image, or by putting together the optoacoustic image and the ultrasonic imaging side by side (or one on top of the other). The image display 40 displays an image based on the composite image data generated by the image merger 311.

The image merger 311 may output the optoacoustic image data or the ultrasonic image data as it is to the image display 40.

The controller 312 transmits a wavelength control signal to the light source driver 102. On receiving the wavelength control signal, the light source driver 102 chooses either the light sources 103A or the light sources 103B. The controller 312 then transmits a light trigger signal to the light source driver 102, which then transmits a drive signal to whichever of the light sources 103A and the light sources 103B is chosen.

In response to an instruction from the controller 312, the transmission control circuit 313 transmits a drive signal to the acoustoelectric converter 202 to make it generate an ultrasonic wave. The controller 312 also controls the reception circuit 301, etc.

The storage 314 is a storage device in which the controller 312 stores various kinds of data, and is configured as a non-volatile memory device, a HDD (hard disk drive), or the like.

Here, it is assumed that the light sources 103A and 103B emit light of different wavelengths. The wavelengths can be set at wavelengths at which a test target exhibits a high absorptance. For example, the wavelength of the light source 103A can be set at 760 nm, at which oxidized hemoglobin in blood exhibits a high absorptance, and the wavelength of the light source 103B can be set at 850 nm, at which reduced hemoglobin in blood exhibits a high absorptance. In this case, for example, when light is emitted from the light source 103A so that the tested object 150 is irradiated with light of a wavelength of 760 nm, the light is absorbed by oxidized hemoglobin contained in blood present in arteries, tumors, etc. inside the tested object 150, and as optoacoustic wave is generated as a result; the optoacoustic image constructor 307 thus generates an optoacoustic image showing the arteries, tumors, etc.

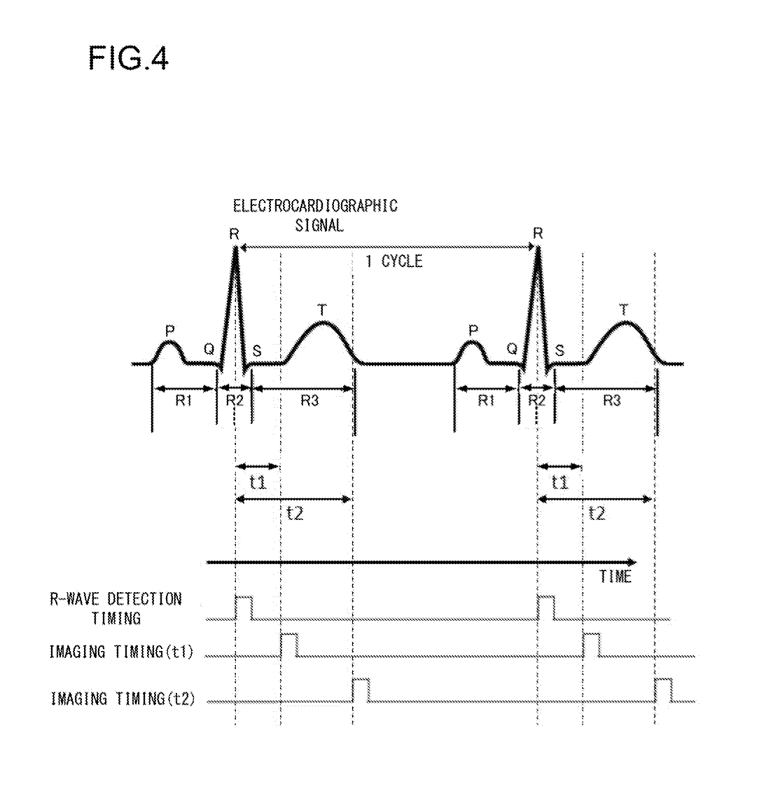

Next, a synchronous electrocardiographic imaging function according to this embodiment will be described with reference also to a timing chart in FIG. 4.

As shown in FIG. 1B, to the optoacoustic imaging device 100 can be externally connected an electrocardiographic detector 110 for detecting an electrocardiographic signal (an example of an organ pulsation signal) of a tested object 150 (human body) from an electrode attached to it. It should be noted that the two tested objects 150 shown at separate places in FIG. 1B for convenience' sake are actually a single entity.

For example as shown in FIG. 4, a normal electrocardiographic signal comprises a p-wave, a q-wave, an r-wave, an s-wave, and a t-wave along the horizontal line, which represents time. In FIG. 4, a region R1 spanning from the start of the p-wave to the start of the q-wave (a so-called pq interval) represents the period from the start of atrial activation to the start of ventricular activation via the atrioventricular junction. A region R2 spanning from the start of the q-wave to the end of the s-wave (a so-called qrs wave) represents the activation of the left and right ventricular muscles (it thus represents cardiac systole). A region R3 spanning from the end of the s-wave to the end of t-wave represents the process of the activated ventricular muscles relaxing (it thus represents cardiac diastole).

The controller 312 acquires the electrocardiographic signal detected by the electrocardiographic detector 110. When the controller 312 detects an r-wave in the acquired electrocardiographic signal, from that timing (r-wave detection timing in FIG. 4) it starts to count time until, at the timing that the controller 312 has counted a predetermined delay time t1 (imaging timing (t1) in FIG. 4), it transmits a light trigger signal to the light source driver 102. In response, for example, the light source drive circuit 102A drives the light source 103A to shine pulsating light on the tested object 150. Then, based on the detection signal of the optoacoustic wave detected by the acoustoelectric converter 202, the optoacoustic image constructor 307 generates optoacoustic image data (still image information). The thus generated optoacoustic image data (first optoacoustic image data) is stored in the storage 314 by the controller 312.

Moreover, at the timing that the controller 312 has counted a predetermined delay time t2 longer than the delay time t1 (imaging timing (t2) in FIG. 4), it transmits a light trigger signal to the light source driver 102. In response, for example, the light source drive circuit 102A drives the light source 103A to shine pulsating light on the tested object 150. Then, based on the detection signal of the optoacoustic wave detected by the acoustoelectric converter 202, the optoacoustic image constructor 307 generates optoacoustic image data (still image information). The thus generated optoacoustic image data (second optoacoustic image data) is stored in the storage 314 by the controller 312. The generation of optoacoustic image data at two different timings as described above is performed every time the r-wave is detected.

The timing delayed by the delay time t1 from the r-wave detection timing allows for a delay in tissue reaction in the tested object 150, and thus corresponds to cardiac systole. The timing delayed by the delay time t2 likewise allows for a delay in tissue reaction in the tested object 150, and thus corresponds to cardiac diastole.

Based on the first and second optoacoustic image data stored in the storage 314, the image display 40 can display the corresponding images (still images) (side by side or otherwise). For example, in a case where the wavelength of the light emitted from the light source 103A used for imaging is set at a wavelength at which oxidized hemoglobin exhibits a high absorptance, if in the images displayed on the image display 40 based on the first and second optoacoustic image data, a high luminance level is observed in a pathologically affected part and a large variation in luminance is observed between the two images, then it is suspected that arterial blood flows into the affected part in synchronism with heart beats, indicating a rather malignant tumor. On the other hand, a small variation in luminance between the two images reveals that the affected part is little affected by heart beats.

Moreover, in this embodiment, within one cycle of an electrocardiographic signal (the period from one r-wave to the next), optoacoustic image data is generated only at two timings corresponding to delay times t1 and t2 respectively, and this helps greatly reduce the amount of data stored in the storage 314. It is however also possible to perform imaging at timings delayed not only by delay times t1 and t2 but also by an intermediate delay time between t1 and t2.

### Second Embodiment

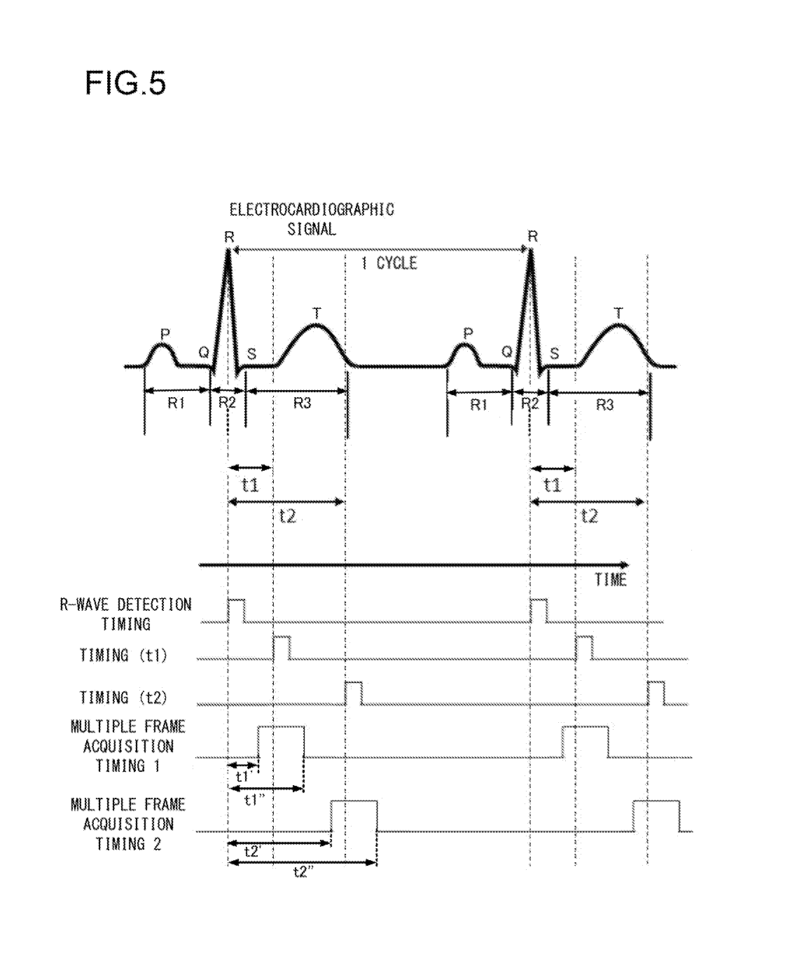

Next, a second embodiment of the present invention will be described. This embodiment is a modified example of the synchronous electrocardiographic imaging function according to the first embodiment. The synchronous electrocardiographic imaging function according to the second embodiment will now be described with reference to a timing chart in FIG. 5.

When the controller 312 detects an r-wave in the electrocardiographic signal acquired from the electrocardiographic detector 110, from that timing (r-wave detection timing in FIG. 5) it starts to count time. At the timing that the controller 312 has counted time corresponding to a predetermined delay time t1′ shorter than the predetermined delay time t1, it starts to transmit a light trigger signal to the light source driver 102. In response, for example, the light source drive circuit 102A starts to drive the light source 103A, and thus the tested object 150 starts to be irradiated with pulsating light. The optoacoustic image constructor 307 then starts to generate optoacoustic image data based on the detection signal of the optoacoustic wave detected by the acoustoelectric converter 202.

The generation of image data by the optoacoustic image constructor 307 is repeated until a predetermined, delay time t1″ longer than the delay time t1 elapses, with a result that optoacoustic image data (first optoacoustic image data) of a plurality of frames is generated and stored in the storage 314.

Moreover, when the controller 312 has counted time corresponding to a predetermined delay time t2′ (longer than the delay time t1″ but shorter than the predetermined delay time t2) from the timing that the r-wave was detected, it starts to transmit a light trigger signal in a similar mariner as described above, so that the optoacoustic image constructor 307 starts generating image generation. The image generation by the optoacoustic image constructor 307 is repeated until a predetermined delay time t2″ longer than the delay time t2 elapses, with a result that optoacoustic image data (second optoacoustic image data) of a plurality of frames is generated and stored in the storage 314.

As described above, in this embodiment, during a period from before to after the time point that a delay time t1 corresponding to cardiac systole lapses, optoacoustic image data (first optoacoustic image data) of a plurality of frames is generated, and during a period from before to after the time point that a delay time t2 corresponding to cardiac diastole lapses, optoacoustic image data (second optoacoustic image data) of a plurality of frames is generated. The generation of image data during two periods as described above is repeated every time an r-wave is detected.

Through the viewing of a plurality of still images displayed on the image display 40 based on the first and second optoacoustic image data stored in the storage 314, a user can easily study the test results in relation to heart beats.

In particular, in this embodiments, even if different tested objects 150 have different tissue reaction delays, it is possible to obtain image data appropriate for conducting diagnosis.

The embodiments through which the present invention is described herein allow for various modifications without departing from the spirit of the present invention. For example, the electrocardiographic detector may be provided in the optoacoustic device.

For another example, the timings of organ pulsation (e.g., heart beats) may be detected by analyzing an optoacoustic image (or ultrasonic image) without using an electrocardiographic signal, and imaging may be performed at the detected timings. This falls within the scope of the present invention.

What is claimed is:

1. An optoacoustic imaging device comprising:
a light source module which irradiates a tested object with light;
a light source driver which drives and controls the light source module;
a detector which detects an optoacoustic wave generated inside the tested object as a result of the tested object being irradiated with the light;
an image generator which generates still image information based on a detection signal from the detector; and
an acquirer which acquires an organ pulsation signal,
wherein the organ pulsation signal is used as a trigger to make the light source driver drive the light source module and to make the image generator generate the still image information.

2. The optoacoustic imaging device according to claim 1,
wherein the image generator generates the still image information only at first and second timings within one cycle of the organ pulsation signal, the first timing corresponding to systole of an organ and the second timing corresponding to diastole of an organ.

3. The optoacoustic imaging device according to claim 2, wherein
the first timing is a timing delayed by a first delay time from a timing at which a predetermined wave indicating contraction of the organ is detected in the organ pulsation signal, and
the second timing is a timing delayed by a second delay time, which is longer than the first delay time, from the timing at which the predetermined wave is detected in the organ pulsation signal.

4. The optoacoustic imaging device according to claim 1,
wherein during a predetermined period from a timing delayed by a predetermined delay time from the timing at which the predetermined wave is detected in the organ pulsation signal, the image generator generates a plurality of sets of still image information.

5. The optoacoustic imaging device according to claim 1,
wherein the light source module comprises a light-emitting diode element.

6. The optoacoustic imaging device according, to claim 1,
wherein the light source module comprises a semiconductor laser element.

7. The optoacoustic imaging device according to claim 1,
wherein the light source module comprises an organic light-emitting diode element.

8. The optoacoustic imaging device according to claim 1,
wherein the organ pulsation signal comprises an electrocardiographic signal.
